(19) United States
(12) Patent Application Publication
(10) Pub. No.: US 2005/0271605 A1
(43) Pub. Date: Dec. 8, 2005

(54) Method of creating images on the body

(75) Inventor: Gina O'Sullivan, Toronto (CA)

Correspondence Address: RICHES, MCKENZIE & HERBERT, LLP, SUITE 1800, 2 BLOOR STREET EAST, TORONTO, ON, M4W 3J5, CA

(73) Assignee: Carre International Inc.

(21) Appl. No.: 11/139,528

(22) Filed: May 31, 2005

Related U.S. Application Data
(60) Provisional application No. 60/576,297, filed on Jun. 3, 2004.

(51) Int. Cl. A61K 7/42

(52) U.S. Cl. 424/59

(57) ABSTRACT

A method of creating an image on the body. In particular, a novel method of creating a tanned image or text on the body using an article of clothing for a mask.

### FIELD OF THE INVENTION

This invention relates to a method of creating an image on the body. In particular, it discloses a novel method of creating a tanned image or text on the body using an article of clothing for a mask.

### SUMMARY OF THE INVENTION

An article of clothing is selected which contains a cut away portion so that the article of clothing can be used as a mask to create a tanned image on the body. A person then puts the article of clothing on his or her body, exposes his or her body to tanning rays either created naturally by the sun or by artificial means or to self-tanning products and the tanning rays or the self-tanning products will then tan the area of the body corresponding to the cut away portion of the article of clothing.

The method is equally applicable using the natural tanning rays of the sun or artificial tanning rays. The person wearing the article of clothing may expose him or her self to the natural tanning rays of the sun or tanning rays of a machine, both of which then expose the portion of the skin under the cut away portion and create the image on the skin.

The method alternatively may involve a die cut patch that may be adhesive that is applied to an area of the body by the user. A sunless tanning solution can then be applied to the die cut area to tan the exposed skin area. When the patch is removed, the tanned image remains.

The method alternatively may involve a patch that can be adhesive and is applied to an area of the body by the user. In an opposite way to the above die cut patch, the skin under the shaped patch is prevented from tanning, leaving an untanned image when the rest of the body becomes tanned. This tanning can be done by natural or artificial means or by self-tanning products. The tanning rays or self-tanning products will thus tan the body except for the part covered by the patch.

This method is particularly suitable in several industries. First, in the advertising industry, clothing or a die cut patch may be made with the company's logo or image in the cut away portion. A person then wears the clothing or patch and creates a tan which is in the form of the company's logo or image. When the person removes the article of clothing or the patch, the company's logo or image is then seen on his or her body. The skin which was exposed to the tanning rays through the cut away portion creates the company's logo or image on the body itself. In the case of the shaped patch, an untanned form of the company's logo or image will be left when the rest of the body is tanned.

An example of such industry is the swimsuit industry. For example, a manufacturer of swimsuits may create a top having its corporate logo or image cut out of the top. The swimmer then wears the top, exposes the exposed skin to tanning rays and therefore creates the corporate logo or image as a tan on the body. When the swimmer competes in competition, the company's logo or image is seen on the body of the swimmer thus creating a new advertising vehicle for the company.

These concepts are equally applicable to the fashion industry. Attractive designs may be created in tops which could be used as masks to transfer the attractive image to the body of the wearer.

These concepts are equally applicable to any industry which wishes to develop a new advertising mode. It can be used on a wide variety of products for men, women and children and can be used with respect to any article of clothing.

One significant advantage of this process is that it causes no harm to the body and leaves no indelible image. The image or logo is created by tanning and of course the tan will “wear off” and can be repeated or changed as desired.

The process may also be used with self-tanning products that can be applied to the skin to create tan colour. These products are available commercially in drug stores and the like and when applied to the skin, create an artificial tan. The products can be applied by sprays, creams or lotions and the like and using the article of clothing as a mask or the die cut patch will create a tan in the cut away portion. The products could also leave an untanned portion when using the shaped patch.

### BRIEF DESCRIPTION OF THE DRAWINGS

FIG. 1 is an article of clothing with a cut away portion on the chest;

FIG. 2 is a die cut patch; and

FIG. 3 is a tanned body after using the product.

### DETAILED DESCRIPTION OF THE INVENTION

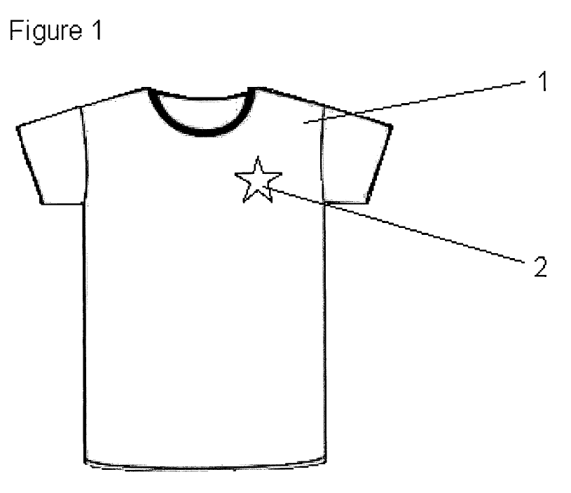

Reference is now made to FIGS. 1 to 3 to illustrate the embodiments of the invention. FIG. 1 shows a t-shirt 1 which contains a cut away image 2 on the chest. Image 2 is shown as a star shape but could be any desired shape. The cut away image 2 allows the exposed body underneath to become tanned.

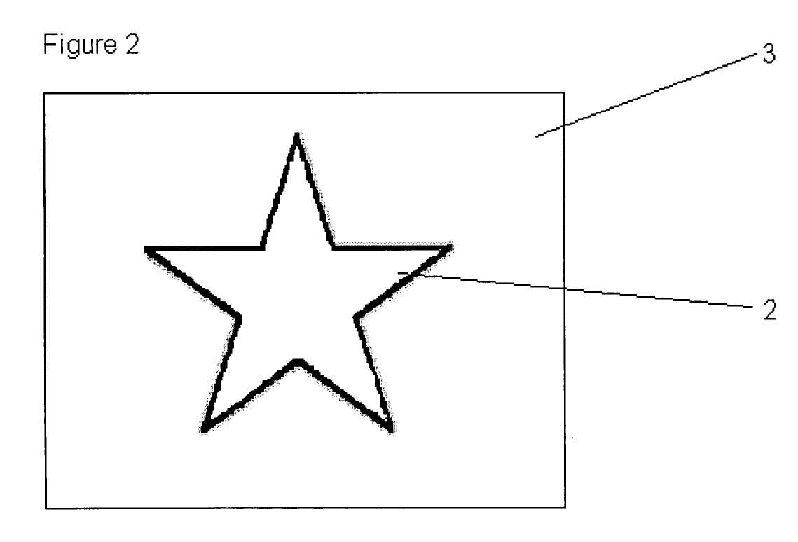

FIG. 2 shows a die cut patch 3 with the cut away image 2. The image 2 is again shown as a star but could be any desired shape. The die cut patch could also be made of an adhesive material so it can stick to the body and stay in place. In an alternative embodiment, not shown, the patch could be a shaped patch designed to leave an untanned part of the body in the shape of the image while the rest of the body tans. For example, a star-shaped cut out could be placed on the body preventing the body from being tanned.

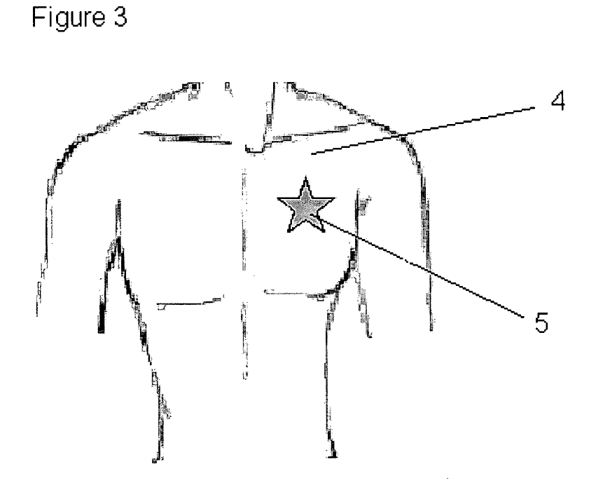

FIG. 3 shows the tanned image 5 on a torso 4 after using the cut away products. If a shaped patch was applied, FIG. 3 would be reserved with the body tanned and the patched area untanned.

1. A method of creating an image on a body which comprises creating a mask comprising as an article of clothing having a cut away portion corresponding to said image, placing the article of clothing on a body, exposing the body to tanning rays.

2. A method of creating an image on a body which comprises a die cut patch having a cut away portion corresponding to said image, placing the die cut patch on a body, exposing the body to tanning rays.

3. A method of creating an image on a body which comprises a shaped patch corresponding to said image, placing the shaped patch on a body, exposing the body to tanning rays.

4. A method as claimed in claim 2 or 3 wherein the patch is an adhesive patch.

5. A method as claimed in any one of claims 1 to 4 wherein said tanning rays are natural tanning rays.

6. A method as claimed in any one of claims 1 to 4 wherein said tanning rays are artificial tanning rays.

7. A method as claimed in any one of claims 1 to 4 wherein said tan is created by using a self-tanning product.
